(19) United States
(12) Patent Application Publication
(10) Pub. No.: US 2026/0144462 A1
(43) Pub. Date: May 28, 2026

(54) Method And Device For Measuring Oxygen Saturation In Blood

(71) Applicant: Nitto Denko Corporation, Osaka (JP)

(72) Inventors: Pongsarun Thiamtawan, Bangkok (TH); Usanee Apijuntarangoon, Bangkok (TH); Phichamon Sakdarat, Bangkok (TH); Visit Thaveeprungsriporn, Singapore (SG); Nuttaporn Suphanimitwatsana, Bangkok (TH)

(73) Assignee: Nitto Denko Corporation, Osaka (JP)

(21) Appl. No.: 18/869,467

(22) PCT Filed: May 29, 2023
(86) PCT No.: PCT/SG2023/050377

§ 371 Date: Nov. 26, 2024

(30) Foreign Application Priority Data
May 30, 2022 (SG) 10202250046F

(51) Int. Cl. A61B 5/1455; A61B 5/00; A61B 5/103; G06N 20/20

(52) U.S. Cl. CPC A61B 5/14551; A61B 5/1032; A61B 5/7221; A61B 5/7267; G06N 20/20

(57) ABSTRACT

The present disclosure generally relates to a computerized method (100) and a device for measuring oxygen saturation in a user's blood. Measure (110) PPG signals from one or more different wavelengths of light, each PPG signal comprising pulsatile and non-pulsatile components. Calculate (120), for each PPG signal, a gradient of the non-pulsatile components of the PPG signal with respect to light intensity of the wavelength. Determine (130) the user's skin tone from one or more gradients of the PPG signals and a first data model. Calculate (140) a modulation ratio from the pulsatile and non-pulsatile components of a pair of PPG signals measured from different wavelengths. Select (150) a second data model based on the user's skin tone. Determine (160) the oxygen saturation in the user's blood from the modulation ratio and the selected second data model.

### CROSS REFERENCE TO RELATED APPLICATION(S)

The present disclosure claims the benefit of Singapore Patent Application No. 10202250046F filed on 30 May 2022, which is incorporated in its entirety by reference herein.

### TECHNICAL FIELD

The present disclosure generally relates to measurement of oxygen saturation in blood. More particularly, the present disclosure describes various embodiments of a method and a device for measuring the oxygen saturation in a user's blood.

### BACKGROUND

Oxygen saturation in blood, or SpO2 level, can be used to detect various health conditions or disorders. For example, Obstructive Sleep Apnea (OSA) is a sleep-related breathing disorder that usually happens when part or all of the upper airway is blocked during sleep. This leads to a reduction in the SpO2 level which can fall by as much as 40% or more in severe cases. Studies have also found correlations between OSA and other conditions and diseases such as cardiovascular disease (CVD), diabetes, mental stress, etc. SpO2 level can also be used as a key indicator for respiratory diseases such as COVID-19.

SpO2 level is commonly measured using a pulse oximeter but this tends to overestimate the SpO2 level for people with dark skin. The pulse oximeter may show a normal SpO2 level but the true SpO2 level could be lower. The user would thus not be aware that he/she is suffering from low SpO2 level and this can be dangerous as there is higher risk of hypoxemia, i.e. low blood oxygen.

A recent study showed that patients with darker skin had nearly three times the frequency of occult hypoxemia that was not detected by pulse oximetry in patients with lighter skin. Occult hypoxemia is a condition wherein the arterial oxygen saturation is less than 88% despite an oxygen saturation of 92% to 96% on pulse oximetry. Since pulse oximetry is widely used for medical decision making, reliance on pulse oximetry to triage patients and adjust supplemental oxygen levels may place patients with darker skin at increased risk of hypoxemia.

Therefore, in order to address or alleviate at least one of the aforementioned problems and/or disadvantages, there is a need to provide an improved method and device for measuring oxygen saturation in blood.

### SUMMARY

According to a first aspect of the present disclosure, there is a computerized method and a measurement device for measuring oxygen saturation in a user's blood. The method comprises: measuring a set of photoplethysmography (PPG) signals from one or more different wavelengths of light, each PPG signal measured from a respective wavelength and comprising pulsatile and non-pulsatile components; calculating, for each PPG signal, a gradient of the non-pulsatile components of the PPG signal with respect to light intensity of the respective wavelength; determining the user's skin tone from one or more gradients of the set of PPG signals and a first data model; calculating a modulation ratio from the pulsatile and non-pulsatile components of a pair of PPG signals measured from a pair of different wavelengths of light; selecting one from a plurality of second data models based on the user's skin tone; and determining the oxygen saturation in the user's blood from the modulation ratio and the second data model selected for the user's skin tone.

According to a second aspect of the present disclosure, there is a computerized method and a measurement device for determine a user's skin tone. The method comprises: measuring a set of photoplethysmography (PPG) signals from one or more different wavelengths of light, each PPG signal measured from a respective wavelength and comprising pulsatile and non-pulsatile components; calculating, for each PPG signal, a gradient of the non-pulsatile components of the PPG signal with respect to light intensity of the respective wavelength; determining the user's skin tone from one or more gradients of the set of PPG signals and a first data model.

A method and device for determining skin tone and measuring oxygen saturation in blood according to the present disclosure is thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIG. 1 illustrates a flowchart of a method for measuring oxygen saturation in a user's blood using PPG signals.

FIGS. 2A to 2E illustrate relationships between light intensity and different wavelengths of light for measuring the PPG signals.

FIGS. 3A to 3L illustrate performance of the different wavelengths of light in determining skin tone.

FIGS. 4A and 4B illustrate relationships between blood oxygen saturation and modulation ratio.

FIGS. 5A and 5B illustrate a pulse verification process performed on the PPG signals.

FIGS. 6A and 6B illustrate the pulse verification process based on motion strength of the PPG signals.

FIGS. 7A and 7B illustrate the pulse verification process based on signal strength of the PPG signals.

FIGS. 8A to 8E illustrate the pulse verification process based on morphology features of the PPG signals.

FIG. 9 illustrates a flowchart of an iterative process for optimizing heart rate boundary for the pulse verification process.

FIG. 10 illustrates a flowchart of the method for measuring blood oxygen saturation and including the pulse verification process.

FIGS. 11A to 11D illustrate performance results of the method for measuring blood oxygen saturation.

### DETAILED DESCRIPTION

For purposes of brevity and clarity, descriptions of embodiments of the present disclosure are directed to a method and device for determining skin tone and measuring oxygen saturation in blood, in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of aspects of particular embodiments. In a number of instances, well-known systems, methods, procedures, and components have not been described in detail so as to not unnecessarily obscure aspects of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment.

As used herein, the terms “a” and “an” are defined as one or more than one. The use of “/” in a figure or associated text is understood to mean “and/or” unless otherwise indicated. The term “set” is defined as a non-empty finite organization of elements that mathematically exhibits a cardinality of at least one (e.g. a set as defined herein can correspond to a unit, singlet, or single-element set, or a multiple-element set), in accordance with known mathematical definitions. The recitation of a particular numerical value or value range herein is understood to include or be a recitation of an approximate numerical value or value range. The terms “first”, “second”, etc. are used merely as labels or identifiers and are not intended to impose numerical requirements on their associated terms.

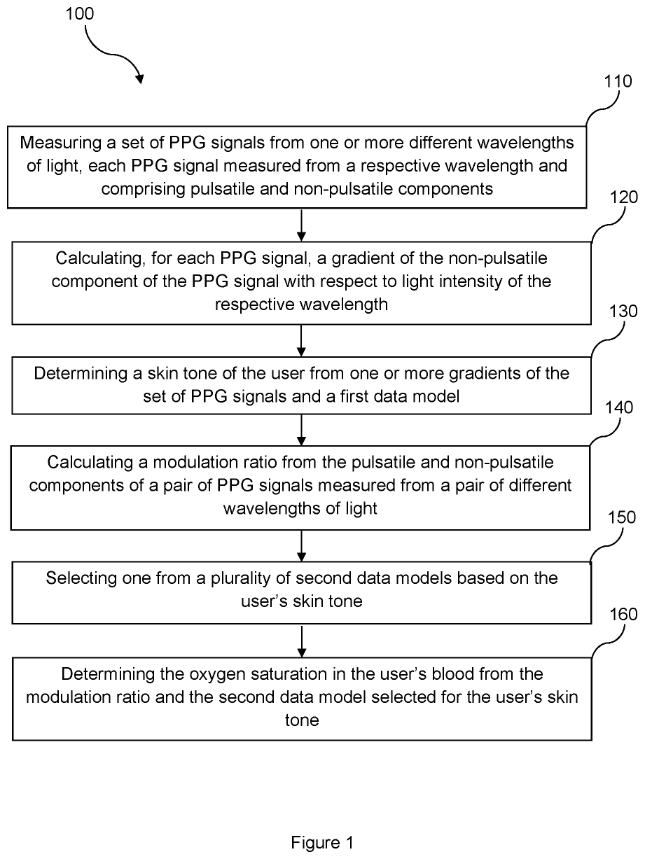

In representative or exemplary embodiments of the present disclosure, there is a computer-implemented or computerized method 100 for measuring oxygen saturation in a user's blood, as shown in FIG. 1. The method can be performed on a measurement device having a processor and various steps of the computerized method are performed in response to non-transitory instructions operative or executed by the processor. The non-transitory instructions are stored on a memory of the measurement device and may be referred to as computer-readable storage media and/or non-transitory computer-readable media. Non-transitory computer-readable media include all computer-readable media, with the sole exception being a transitory propagating signal per se. The measurement device may be a wearable device worn on the user, such as on the wrist or finger, to measure the oxygen saturation in the user's blood.

The method 100 includes a step 110 of measuring a set of photoplethysmography (PPG) signals from one or more different wavelengths of light. In many embodiments, the set of PPG signals includes a plurality of PPG signals. The step 110 includes measuring the plurality of PPG signals from a plurality of different wavelengths of light. The PPG signals are obtained from the amount of light absorption by inverting the light intensity with a photodetector after the light is transmitted through or reflected from human tissue. For example, the measurement device includes lighting elements (e.g. LEDs) for emitting the one or more or the plurality of wavelengths of light, as well as photodetectors for detecting the one or more or the plurality of wavelengths of light, such as light that has reflected off the user's skin. The one or more or plurality of different wavelengths of light may define at least one wavelength in the range of 495 nm to 1000 nm.

Each PPG signal is measured from a respective wavelength or colour of light, such as but not limited to green, orange, red, and infrared. For example, the measurement device may include four lighting elements for emitting green, orange, red, and infrared light. The wavelength for green light may range from 495 to 570 nm, preferably with a peak wavelength of 536 nm. The wavelength for orange light may range from 570 to 620 nm, preferably with a peak wavelength of 610 nm. The wavelength for red light may range from 620 to 740 nm, preferably with a peak wavelength of 660 nm. The wavelength for infrared light may range from 780 to 1000 nm, preferably with a peak wavelength of 950 nm. The plurality of wavelengths may be selected from the range 495 nm to 1000 nm, wherein the wavelengths may be at least 50 nm apart from each other.

Each PPG signal includes pulsatile and non-pulsatile components. The pulsatile component, also known as the alternating current (AC) component, is related to changes in arterial blood volume and is synchronized with the cardiac cycle. The non-pulsatile component, also known as the direct current (DC) component, refers to the remainder of the PPG signal excluding the pulsatile component. The pulsatile component is superimposed on the non-pulsatile component in the PPG signal. The non-pulsatile component is related to the level of light absorption by the tissue, bones, venous blood, and skin pigments.

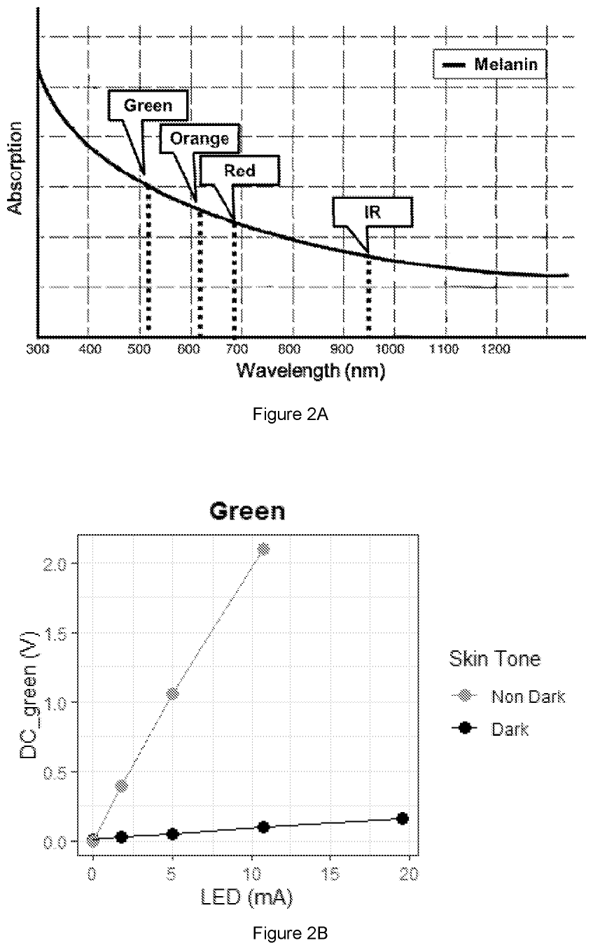

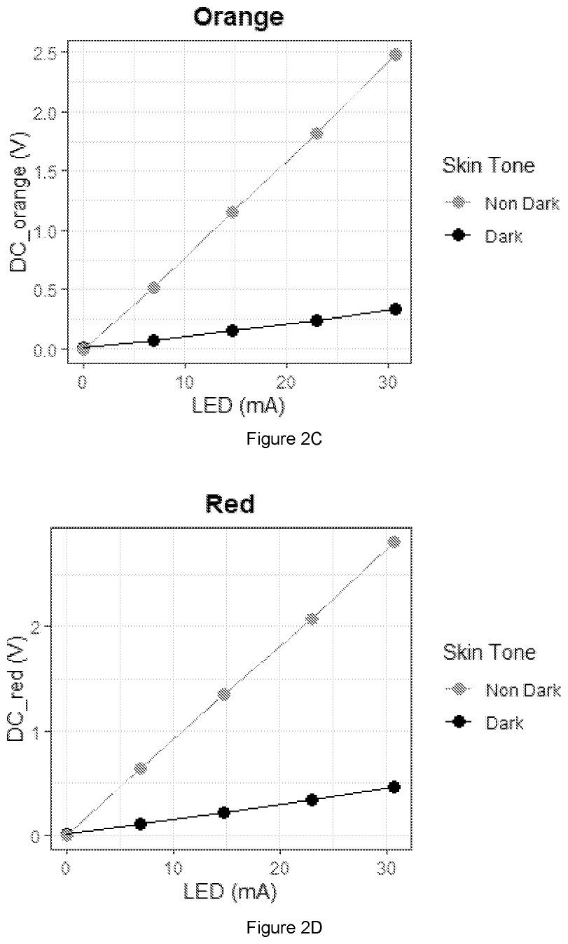

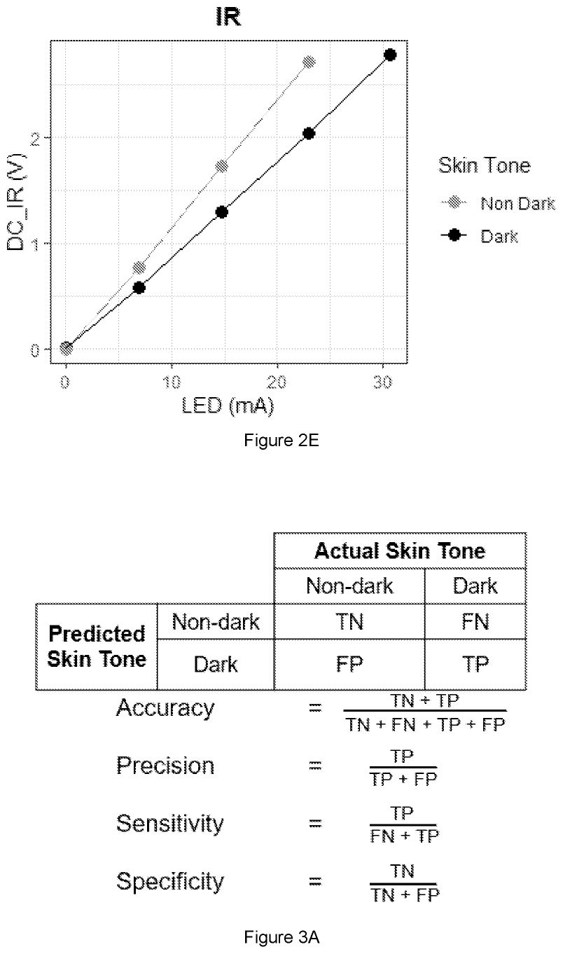

It was found that the non-pulsatile component increases when the light intensity increases, in an approximately linear relationship. Moreover, for the same wavelength of light and the same increase in light intensity, the non-pulsatile component increases more for people with light or non-dark skin compared to people with dark skin. This is because light absorption by the skin is affected by skin pigments such as melanin. Darker skin pigments, i.e. more melanin, absorbs light more than lighter skin pigments, resulting in less light being reflected from the skin and detected by the photodetector, hence a smaller non-pulsatile component in the PPG signal. For the same skin pigments, the light absorption is different for different wavelengths of light. For example as shown in FIG. 2A (extracted from González-Rodriguez et al, Current Indications and New Applications of Intense Pulsed Light, Actas Dermo-Sifiliográficas, 2015), melanin skin pigment absorbs green light the most and infrared light the least.

FIGS. 2B to 2E show the relationships between the non-pulsatile components and the light intensity for four wavelengths of light—green, orange, red, and infrared. Each of FIGS. 2B to 2E shows the relationships for two skin tones—light or non-dark skin tone, and dark skin tone. The skin tones may be classified according to the Fitzpatrick skin typing scale, wherein Types I-III fall under the non-dark skin tone and Types IV-VI fall under the dark skin tone. The skin tones may also be classified according to Monk skin tone scale, wherein Monk 01-05 fall under the non-dark skin tone and Monk 06-10 fall under the dark skin tone. The skin tones may also be classified into more than two tones, for example, up to all six tones of the Fitzpatrick skin typing scale. The skin tones may be classified according to other scales, such as the Von Luschan's chromatic scale that classifies skin colours.

As shown in FIGS. 2B to 2E, the non-pulsatile component is defined by the DC level measured in volts, and the light intensity is defined by the electric current to the lighting element measured in amperes. The relationships are established using a free drive process wherein the electric current is incremented in steps and the DC level is measured for each increment step of the electric current. The rate of increase of the DC level with respect to the light intensity is then calculated as the gradient or slope. Notably, for the same wavelength, the gradient is larger for the non-dark skin group compared to the dark skin group. The gradients are different for different wavelengths due to the differences in absorption ability, as shown in FIG. 2A. The gradient is thus affected by at least two factors—skin type and wavelength.

Pre-collected data from multiple subjects about the gradients, wavelengths, and skin tones are used to construct a first data model using one or more statistical and/or machine learning algorithms. Preferably, the first data model is constructed using classification algorithms and/or regression analysis such as logistic regression. Alternatively, the first data model can be constructed using other algorithms or mathematical models such as decision trees and random forests.

The method 100 includes a step 120 of calculating, for each PPG signal measured in the step 110, the gradient of the non-pulsatile components of the PPG signal with respect to light intensity of the respective wavelength. The gradients of each wavelength may be normalized by a gradient of one wavelength, summation of at least two wavelengths, or Euclidean norm of gradients of at least two wavelengths. The method 100 includes a step 130 of determining the user's skin tone from the gradients of the set of PPG signals and the first data model. The step 130 includes determining the user's skin tone from one or more gradients of the set of (one or more) PPG signals and the first data model. In many embodiments, the step 130 includes determining the user's skin tone from two or more gradients of the plurality of PPG signals and the first data model.

Notably, one or more gradients from one or more different wavelengths are used to differentiate the skin tones and determine the user's skin tone (i.e. dark or non-dark) from the first data model. In some embodiments, one gradient from a single wavelength of light, such as green, orange, red, or infrared light, is used to determine the user's skin tone. Preferably, the wavelengths of light for measuring the PPG signals define at least one of green, orange, red, and infrared light. In some embodiments, multiple gradients from multiple wavelengths of light, such as green, orange, red, or infrared light, are to determine the user's skin tone. Preferably, the wavelengths of light for measuring the PPG signals define at least two of green, orange, red, and infrared light. In one embodiment, two gradients from orange and infrared light are used to determine the user's skin tone. In another embodiment, three gradients from green, orange, and infrared light are used to determine the user's skin tone.

Tests were done to evaluate the different wavelengths, gradients, and skin tones. 49 subjects with different skin tones participated in these experiments. These 49 subjects included 32 subjects with non-dark skin, 16 subjects with dark skin, and 1 subject with dark skin on the right hand and non-dark skin on the left hand. 98 skin tone datapoints were obtained from both hands of the 49 subjects. 15 different combinations of one to four wavelengths of light were tested in 500 iterations, each combination having one to four different wavelengths. In each iteration, 15 subjects were randomly selected as the test dataset (with 30 skin tone datapoints) and the other 34 subjects form the training dataset (with 68 skin tone datapoints). The 15 combinations are listed as follows.
1. Green.
2. Red.
3. Infrared.
4. Orange.
5. Green and red.
6. Green and infrared.
7. Green and orange.
8. Red and infrared.
9. Red and orange.
10. Infrared and orange.
11. Green, red, and infrared.
12. Green, red, and orange.
13. Green, infrared, and orange.
14. Red, infrared, and orange.
15. Green, red, infrared, and orange.

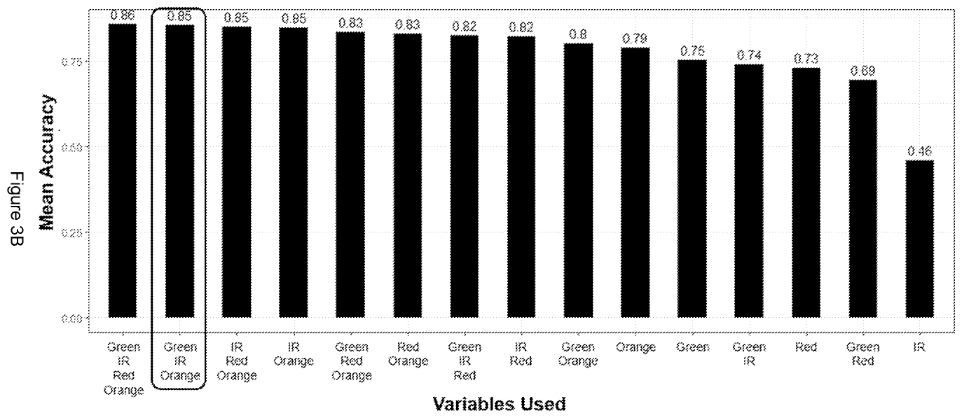

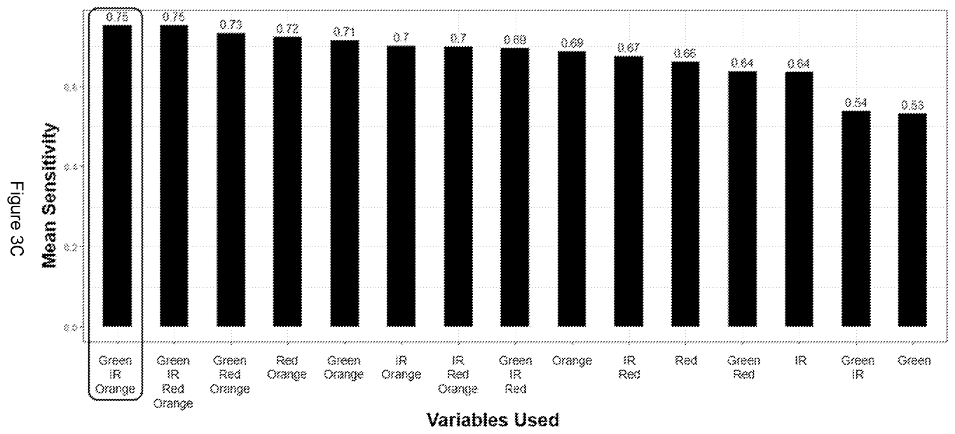

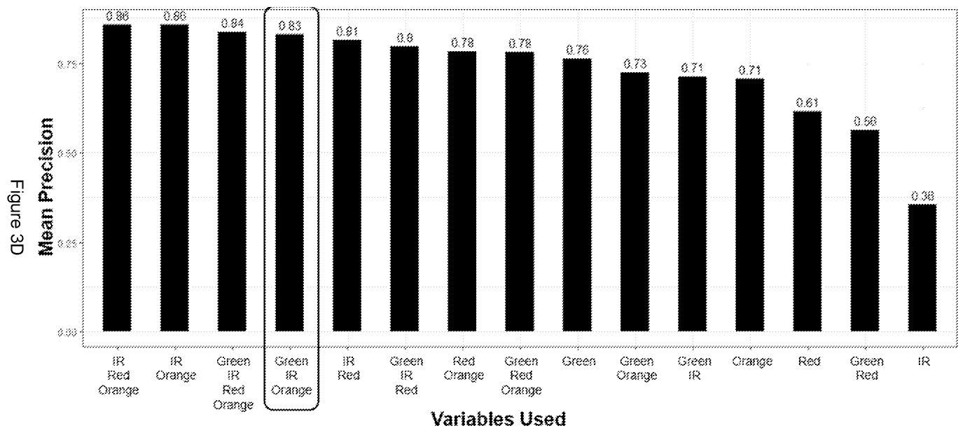

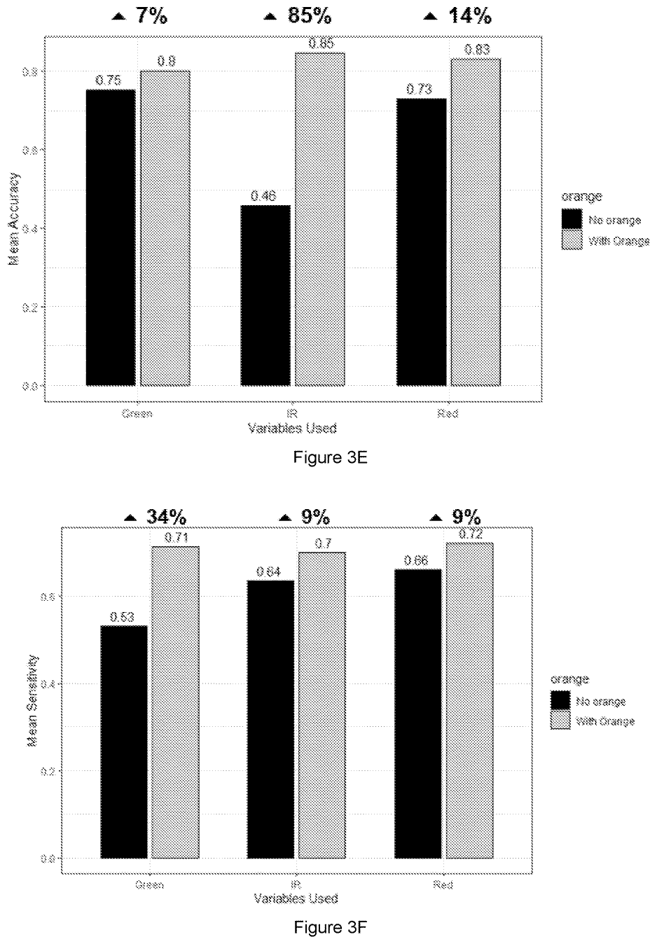

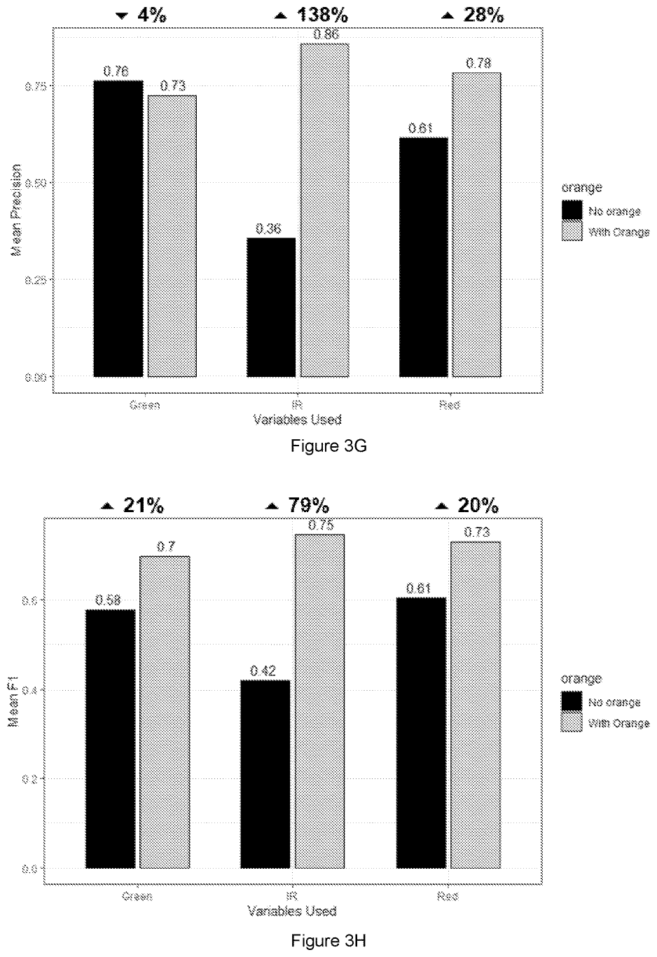

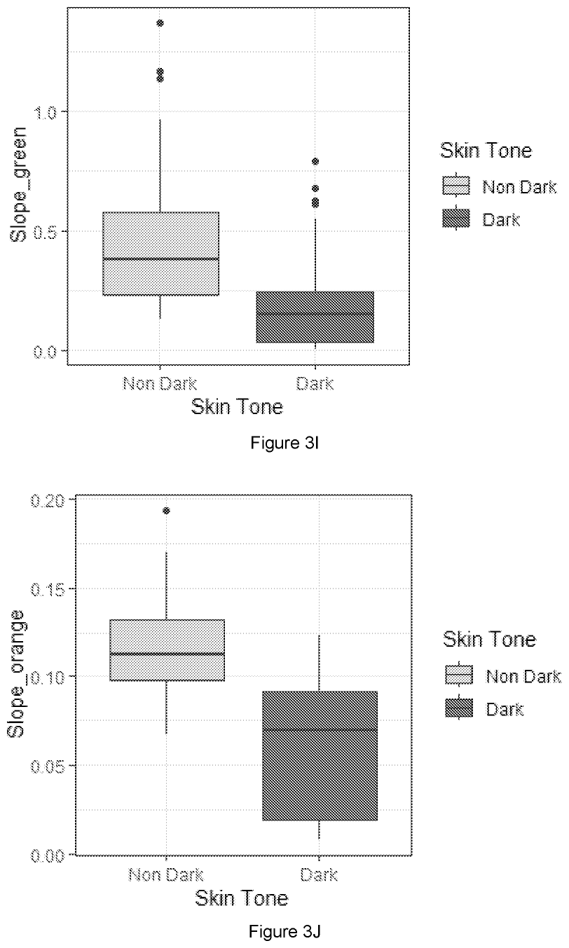

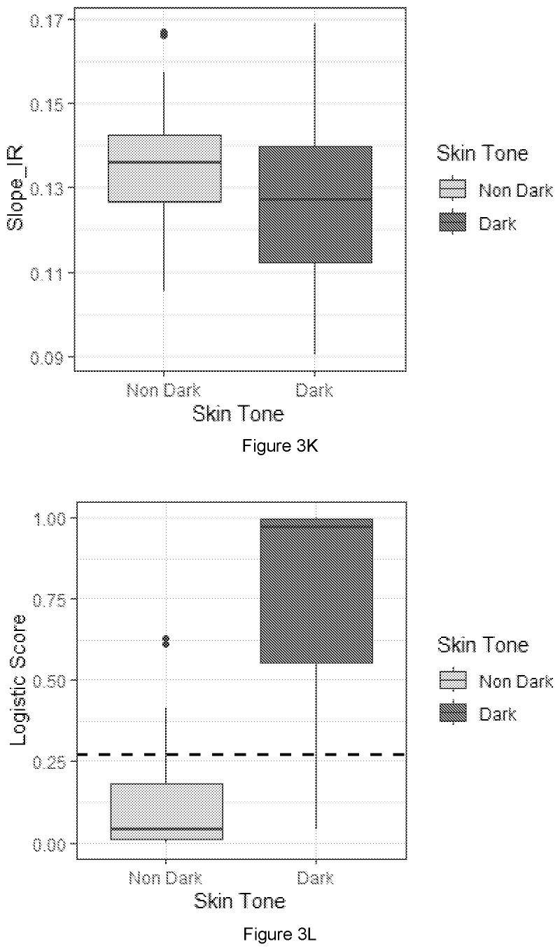

The training dataset was used to train the first data model using logistic regression and the trained first data model was used to evaluate performance on the test dataset in determining skin tones. In the evaluation, four performance indicators—accuracy, precision, sensitivity, and specificity—were calculated as shown in FIG. 3A. Dark skin tone is defined as positive and non-dark skin tone is defined as negative. TN means true negative and refers to the number of correct predictions of actual non-dark skin tone datapoints. TP means true positive and refers to the number of correct predictions of actual dark skin tone datapoints. FP means false positive and refers to the number of actual non-dark skin tone datapoints which were predicted as dark skin tone by the first data model. FN means false negative and refers to the number of actual dark skin tone datapoints which were predicted as non-dark skin tone by the first data model.

After completing the 500 iterations, the mean and standard deviation of each performance indicator was calculated. The results on the accuracy, sensitivity, and precision performance indicators are shown in FIGS. 3B to 3D. It was observed that using more different wavelengths of light achieved better performance. A single wavelength of light may yield good performance such as if it is orange light. However, using four different wavelengths of light would require more LEDs in the measurement device and lead to higher capacity requirements and costs. An optimal combination would be to use three different wavelengths of light to achieve good performance (i.e. good balance and optimization of accuracy, sensitivity and precision), while reducing the number of LEDs. It was found that the optimal combination is green, orange, and infrared light.

Additionally, it was observed that the inclusion of orange light to the other colours (green, red, and infrared) improved the performance of determining the skin tone. The results for green, red, and infrared light with and without orange light are shown in FIGS. 3E to 3H. FIG. 3H shows the F1 scores which is an overall performance indicator based on the harmonic mean of the sensitivity and precision indicators, as shown below. The F1 score improved by at least 20% when orange light is used together with either green, red, or infrared light.

$$F 1 = \frac{2}{( \frac{1}{Sens itivity} ) + ( \frac{1}{Prec i s i o n} )}$$

The user's skin tone is attributed to the melanin content of the user's skin and different melanin content absorbs light to different extents, with more melanin absorbing more light. To differentiate skin tone, a suitable wavelength of light should penetrate to the depth of skin where melanin is, has good absorptivity by melanin, and varies according to melanin content. Wavelengths around the green to orange spectrum have good penetration and are well absorbed by melanin, as shown in FIG. 2A. Hence, these colours are suitable for differentiating skin tone. In addition, orange light penetrates deeper into the skin than green light, and is better able to reach the depth where melanin is. As reflected by the results, the use of orange light as the single wavelength of light, or the inclusion of orange light in a combination of two or more different wavelengths of light, provided good performance in determining skin tone.

The optimal combination of green, orange, and infrared wavelengths yielded the best performance in determining the skin tone based on the gradients without using too many LEDs and achieving a good balance and optimization of accuracy, sensitivity and precision. The boxplots of gradients and skin tones for each wavelength in this combination are shown in FIGS. 3I to 3L. Notably, the gradients are larger for non-dark skin tone compared to dark skin tone.

The first data model was trained using logistic regression for two skin tones (dark and non-dark) and the output for logistic regression is logistic scores. A logistic score is a probability to be of dark skin tone. A skin tone threshold is defined to separate the dark and non-dark skin tones. For example, the skin tone threshold can be defined by maximizing the F1 score.

When the logistic score P(Dark) of the user calculated from the first data model is greater than or equal to the skin tone threshold X, the user would be classified as dark skin tone, as shown below.

$$Predicted Skin Tone = \{ \begin{matrix}Dark ( 1 ) & if P ( Dark ) \geq X \\ Nondark ( 0 ) & if P ( Dark ) < X\end{matrix}$$

The logistic score P(Dark) can be calculated using the equation below. M_{G} is the gradient value for green light, M_{O} is the gradient value for orange light, and M_{IR} is the gradient value for infrared light. a, b, c, and d are constants.

$$P ( Dark ) = sigmoid [ a + b ( {M}_{G} ) + c ( {M}_{O} ) + d ( {M}_{I R} ) ]$$

FIG. 3L shows the boxplot of logistic score and skin tone for the combination of green, orange, and infrared wavelengths. The dashed line represents the skin tone threshold X which may range from 0.1 to 0.7. In an exemplary experiment, this was calculated to be around 0.27 for the combination of green, orange, and infrared wavelengths. This value may change with more data becoming available. Notably, the logistic scores of non-dark and dark skin tones are almost completely separated, indicating that this combination of wavelengths can reliably determine the user's skin tone as dark or non-dark skin tone. It will be appreciated that the skin tone thresholds will be different for each wavelength or various combinations of at least two different wavelengths. It will also be appreciated that the first data model can be trained for determining the skin tone using a single wavelength of light.

The method 100 includes a step 140 of calculating a modulation ratio from the pulsatile and non-pulsatile components of a pair of PPG signals measured from a pair of different wavelengths of light. The modulation ratio is also known as the R ratio. The pair of different wavelengths preferably define red and infrared light. The modulation ratio is defined as the ratio of a first quotient to a second quotient. The first quotient is derived from the pulsatile and non-pulsatile components (i.e. AC/DC) from the first wavelength of light, such as red light. The second quotient is derived from the pulsatile and non-pulsatile components (i.e. AC/DC) from the second wavelength of light, such as infrared light. The modulation ratio or R ratio can be defined as follows.

$$R ratio = \frac{( \frac{{AC}_{R}}{{DC}_{R}} )}{( \frac{{AC}_{IR}}{{DC}_{IR}} )}$$

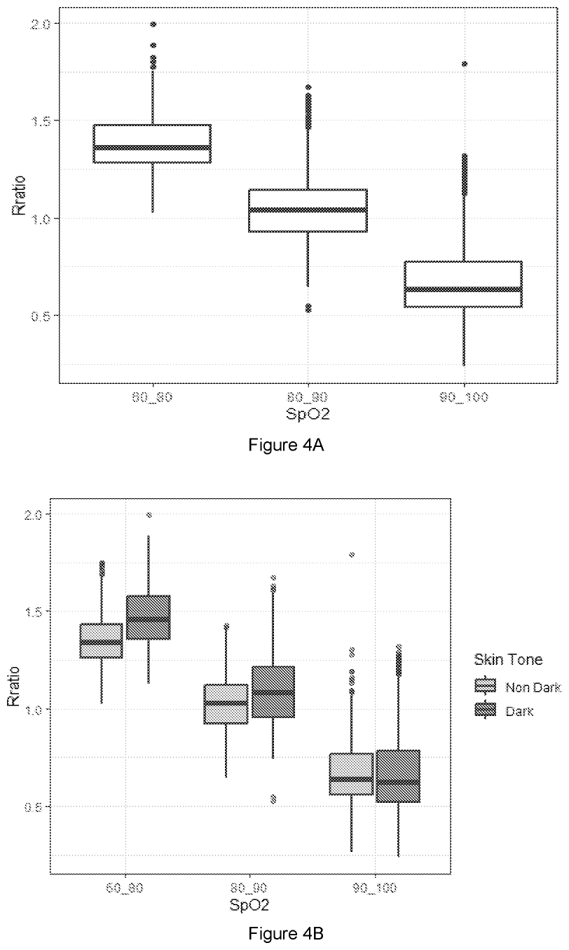

As shown in FIG. 4A, the oxygen saturation in blood (or SpO2 level) is negatively correlated with the modulation ratio. Notably, the SpO2 level increases when the modulation ratio decreases, but their relationships are different for different skin tones. As shown in FIG. 4B, for the same increase in modulation ratio, the SpO2 level decreases slightly more for dark skin compared to non-dark skin.

The method 100 includes a step 150 of selecting one from a plurality of second data models based on the user's skin tone. The method 100 includes a step 160 of determining the oxygen saturation in the user's blood from the modulation ratio and the second data model selected for the user's skin tone.

Other parameters may be used in addition to the modulation ratio to determine the blood oxygen saturation, such as the first quotient or second quotient as shown below. Alternatively or additionally, various mathematical functions may be applied to the modulation ratio and/or any of the parameters used in determining the blood oxygen saturation, such as logarithmic, square root, etc.

$$( R ratio ) + ( \frac{{AC}_{R}}{{DC}_{R}} ) \\ ( R ratio ) + ( \frac{{AC}_{IR}}{{DC}_{IR}} )$$

The second data models are constructed according to the different skin tones, such that there is a second data model for each skin tone. The second data models include a model for dark skin tone and a model for non-dark skin tone. Pre-collected data from multiple subjects about the SpO2 levels, modulation ratios, and skin tones are used to construct the second data models using one or more statistical and/or machine learning algorithms. Preferably, the second data models are constructed using classification algorithms and/or regression analysis, such as linear regression or polynomial regression. Alternatively, the second data models can be constructed using other algorithms or mathematical models such as support vector machine.

A training dataset of PPG signals measured from red and infrared wavelengths was used to train the second data models using regression analysis. The PPG signals was subjected to a pulse verification process 200 to improve the quality of the PPG signals, as described further below. The PPG signals were measured in a set of 4-second time periods and features such as modulation ratio and signal strength were calculated for each time period. PPG signals in a time period may be rejected if they do not meet predefined criteria, such as if the modulation ratio is below a threshold value (e.g. from 2.0 to 3.0 or preferably around 2.3), the signal strength from the red PPG signal is below a threshold value (e.g. from 0.2 to 1.5 or preferably around 0.75), and/or the signal strength from the infrared PPG signal is below a threshold value (e.g. from 0.2 to 1.5 or preferably around 0.67). The passed PPG signals are then used to train the second data models using the modulation ratio as an independent variable and SpO2 level as a dependent variable.

The training dataset was split into a dark and non-dark datapoints based on the skin tone predicted by the first data model. The dark datapoints were used to train one second data model for dark skin tone, and the non-dark datapoints were used to train another second data model for non-dark skin tone. The respective second data model is selected based on the user's skin tone and can be used to measure or predict the user's SpO2 level from the modulation ratio, as shown in the equations below. w, x, y, and z are coefficients derived from the second data models.

$$Predicted SpO 2 ( Dark ) = w - x ( R ratio ) \\ Predicted SpO 2 ( Nondark ) = y - z ( R ratio )$$

It will be appreciated that the first data model can be constructed with finer variations or distinctions across skin tones, such as three or more skin tones, and the second data models can be constructed according to the number of skin tones. Finer skin tone classifications can more accurately determine the user's skin tone and improve measurement of the user's SpO2 level based on the user's skin tone. For example, the skin tones can be classified into all of Types I-VI of the Fitzpatrick scale, or all 36 categories of the Von Luschan's chromatic scale.

In addition or alternative to skin tones, the first data model may be constructed to determine the light absorbance, reflectance, and/or transmittance of other skin types or conditions and categorise them into different skin tone categories. Some examples of skin types include hairy/glabrous skin, oily/dry skin, pigmentation on skin (such as moles), or any combination thereof.

The method 100 requires only a few parameters derived from the PPG signals to determine the user's skin tone and subsequently measure the user's blood oxygen level using the first and second data models. The second data models are selected based on the user's specific skin tone so that the SpO2 levels predicted by the selected second data model are more accurate for the user. This addresses the problem of overestimating the SpO2 level for users with dark skin and decreases the risk of hypoxemia.

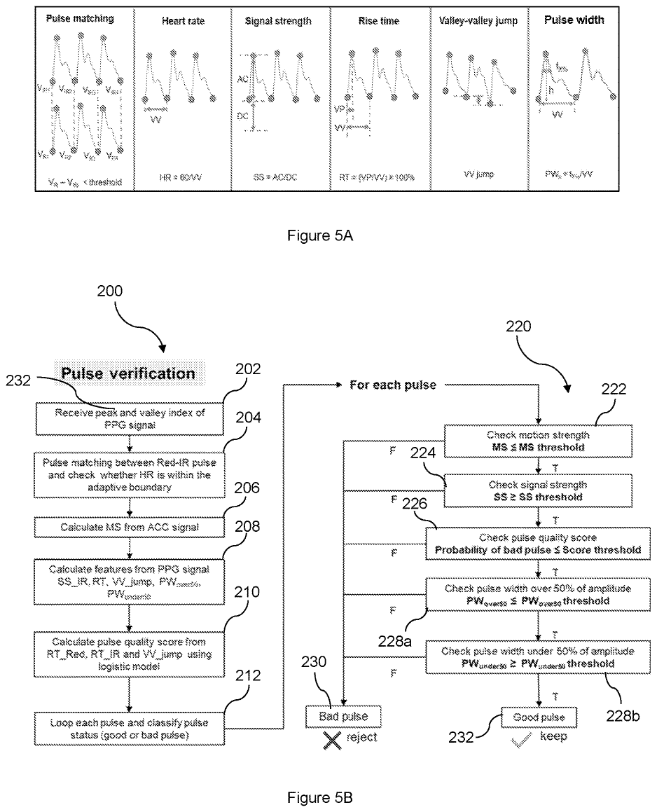

In some embodiments, the method 100 includes performing the pulse verification process 200 on the pair of PPG signals for calculating the modulation ratio. The quality of the PPG signals depends on various factors such as user motion, ambient light, and temperature, and such factors can cause inaccurate measurements of the user's SpO2 level. This might lead to misinterpretation of the SpO2 measurements and cause anxiety to the user. The pulse verification process 200 rejects pulses in the PPG signals based on a third data model, as these pulses can potentially give unreliable or erroneous measurements. For example, the pulse verification process 200 rejects pulses in the PPG signals that do not satisfy conditions defined in the third data model. The pulse verification process 200 ensures that the PPG signals have good quality pulses to measure the user's SpO2 level more accurately. The third data model can be constructed from pre-collected data using one or more statistical and/or machine learning algorithms. Preferably, the third data model is constructed using a comparison of threshold values based on pulse features and classification algorithms and/or regression analysis such as logistic regression. Pulse features include matching difference threshold between PPG signals within a time period, motion strength, and signal strength. Some conditions or pulse features used in the third data model for the pulse verification process 200 are shown in FIG. 5A.

In one example, the conditions in the third data model include a matching difference threshold between the PPG signals within a time period (such as at least 4 seconds), such that the PPG signals are rejected if they do not meet the matching difference threshold. As the pair of PPG signals are measured from two different wavelengths such as red and infrared, the pulsatile components of the pair of PPG signals should be synchronized to improve accuracy of the modulation ratio. For example, the valleys of the pulses in each PPG signal are defined and corresponding pairs of valleys within the time period are compared to each other. The matching difference threshold can be defined as the allowable time limit for corresponding valley pairs that do not match each other. If the corresponding valley pairs in the PPG signals do not occur within the allowable time limit, the valley pairs would be considered as unsynchronized. As the valley pairs have exceeded the matching difference threshold, this specific unsynchronized valley pair would be rejected. Alternatively, the pulses in each PPG signal may be compared using the peaks of the pulses instead of or in addition to the valleys.

In one example, the conditions in the third data model include a motion strength threshold, such that pulses with motion strength above the motion strength threshold are rejected. The motion strength of the pulses is related to noise in the PPG signals which can introduce error in the SpO2 measurements. The motion strength can be determined from an accelerometer signal. The accelerometer signal can be measured by an accelerometer module (which can measure acceleration on one, two, or three axes) in the measurement device that measured the PPG signals. The pulses tend to have high motion strength if the user is moving vigorously while the PPG signals are being measured. The motion strength threshold removes noisy pulses from the high motion parts of the PPG signals.

In one example, the conditions in the third data model include a signal strength threshold, such that pulses with signal strength below the signal strength threshold are rejected. Signal strength of the pulses is defined as the ratio of the pulsatile components to the non-pulsatile components of the PPG signals. A high ratio indicates a strong pulse and there is sufficient degree of the pulsatile component to calculate the modulation ratio accurately.

The motion strength threshold and signal strength threshold thus reject pulses with high noise and low signal strength, resulting in better PPG signals with high signal-to-noise ratio (SNR). This improves accuracy of the modulation ratio and subsequently measurement of the SpO2 level.

In one example, the conditions in the third data model include a bad pulse score threshold which represents the probability threshold of a pulse being a bad pulse or of poor quality, such that pulses scoring above the bad pulse score threshold based on their morphology features are rejected. More specifically, the third data model uses the bad pulse score threshold to verify signal quality of the PPG signals based on the morphology features. Pulses with morphology features that cause the pulses to have a high bad pulse score are rejected.

As shown in FIG. 5A, the morphology features may include a rise time which is the percentage of the valley-to-peak interval to the valley-to-valley interval of a pulse. The morphology features may include a valley-valley jump which is the amplitude difference between the two valleys of a pulse. The morphology features may include a heart rate that estimated from the valley-to-valley interval of a pulse. For example, the conditions in the third data model may include a heart rate boundary, such that pulses are rejected if the corresponding heart rates are outside of the heart rate boundary.

The morphology features may include a pulse width feature derived from the PPG signal. For example, the conditions in the third data model may include an upper pulse width threshold, such that the pulses with an upper pulse width (corresponding to over 50% of systolic amplitude of the pulses) above the upper pulse width threshold are rejected. For example, the conditions in the third data model comprise a lower pulse width threshold, such that the pulses with a lower pulse width (corresponding to under 50% of systolic amplitude of the pulses) below the lower pulse width threshold are rejected.

The pulse width feature (PWx) represents the time interval between x % of the full height (h) of the pulse, the systolic amplitude of the pulse, divided by total time interval of the pulse (from first valley to second valley) as shown in FIG. 5A. In the pulse verification process 200, PWx under 50% (PW_{under50%}) and over 50% (PW_{over50%}) of amplitude are used as features to classify good and bad pulses, respectively. PW_{50%} refers to the pulse width between points corresponding to 50% of the PPG systolic peak amplitude. PW_{under50%} refers to the lower pulse width between points corresponding to x % of the PPG systolic peak amplitude, and this x % ranges from 0% to 50%, preferably from 20% to 30%. PW_{over50%} refers to the upper pulse width between points corresponding to x % of the PPG systolic peak amplitude, and this x % ranges from 50% to 100%, preferably from 70% to 80%.

PW_{over50%} can be used to identify a corrupted PPG signal by unknown noise that might be caused by the inappropriate wearing of the measurement device. At x % ranging from 50% to 100%, the width of the PPG systolic peak amplitude tends to be wider for a corrupted PPG signal compared to a good PPG signal. Pulses with the upper pulse width, i.e. PW_{over50%}, above the upper pulse width threshold are classified as bad pulses and rejected. PW_{under50%} can be used to identify distorted PPG signals resulting from blood vessel congestion caused by the wearing the measurement device too tightly. At x % ranging from 0% to 50%, the width of the PPG systolic peak amplitude tends to be narrower for a distorted PPG signal compared to a good PPG signal. Pulses with the lower pulse width, i.e. PW_{under50%}, below the lower pulse width threshold are classified as bad pulses and rejected.

An exemplary pulse verification process 200 is shown in FIG. 5B. In a step 202, the peaks and valleys of the pulses in the PPG signals are defined. In a step 204, the pulses in the PPG signals within the same time period are compared to each other, such as by their corresponding valley pairs. If the corresponding valley pairs in the PPG signals do not occur within an allowable time limit, the valley pairs would be considered as unsynchronized. As the valley pairs have exceeded the matching difference threshold, this specific unsynchronized valley pair would be rejected. The matching difference threshold may be defined as 0% to 25%, preferably 10% to 25%, of the total number of valley pairs in the PPG signals (i.e. the sampling size).

Additionally, the step 204 may include checking whether the heart rate is within the heart rate boundary. As mentioned above, the heart rate can be estimated from the valley-to-valley interval of a pulse. If the heart rate is outside of the heart rate boundary, then the pulse from which the heart rate is estimated is rejected. The heart rate boundary may be a fixed standard deviation from a mean heart rate determined from a sample dataset of heart rate data, such as within ±10 to ±30 bpm or preferably ±20 bpm. Alternatively, the heart rate boundary may have a variable standard deviation from the mean heart rate.

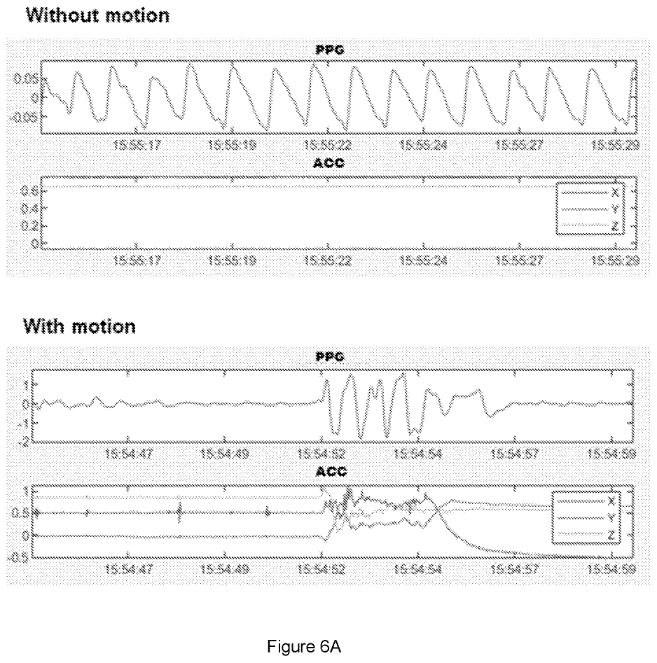

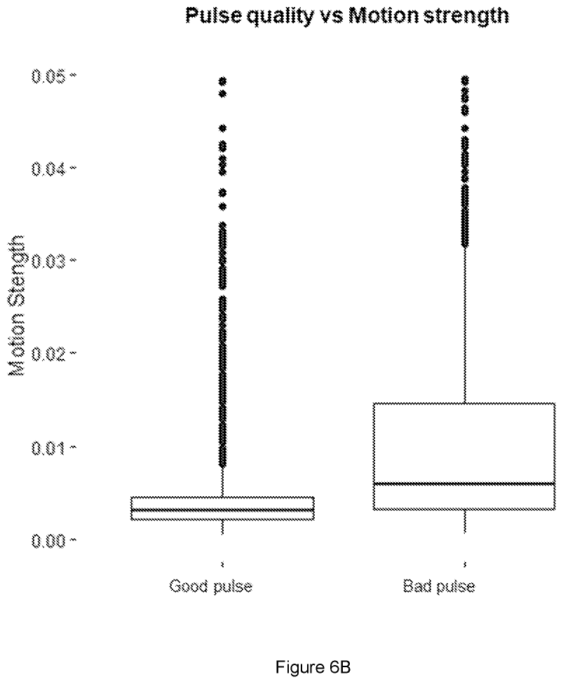

In a step 206, the motion strength of the pulses in the PPG signals is calculated from the accelerometer signal. The motion strength represents the level of motion from the maximum magnitude of the accelerometer signal. FIG. 6A shows the effect of motion (represented by the accelerometer or ACC signal) on the PPG signal. When there is no motion, the shape of pulses in the PPG signal can be defined clearly. When there is motion, the PPG signal is distorted relative to the magnitude of motion, which makes it difficult to define the peaks and valleys of the pulses.

The third data model was trained using a sample dataset of PPG signals and accelerometer signals from 16 subjects. The good and bad pulses were defined based on the motion strength threshold. The motion strength threshold can be defined by maximizing the F1 score, which is the harmonic mean of the sensitivity and precision performance indicators of the third data model for motion strength. The motion strength threshold may range from 0.01 to 0.1, for example 0.018 in this sample dataset. As shown in the boxplot in FIG. 6B, good pulses are associated with low motion strength.

In a step 208, the signal strength of the pulses in the PPG signals is calculated. Preferably, the signal strength of the infrared PPG signal is used. The signal strength determines whether the magnitude of pulses is large enough for the PPG signal to be considered as well-defined.

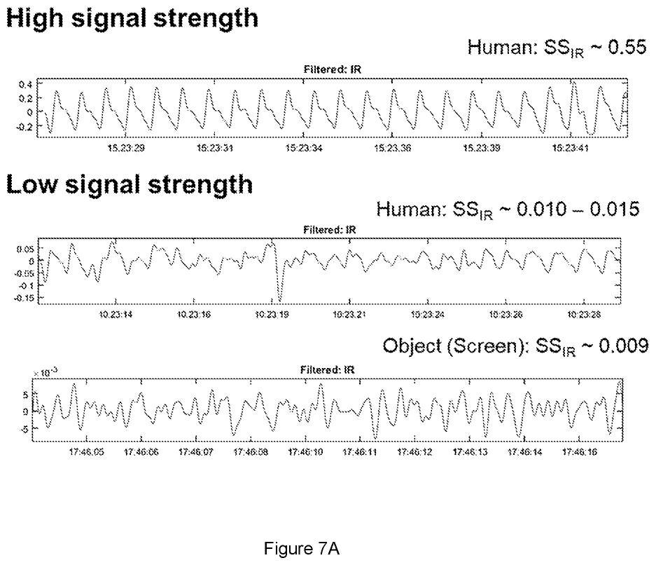

FIG. 7A shows exemplary PPG signals measured from different sources under low to no motion. The first and second PPG signals are measured from a human user and the third PPG signal is measured from an inanimate object. As shown in the boxplot in FIG. 7B, the signal strength of the third PPG signal (around 0.009) is relatively low compared to the first and second PPG signals (around 0.55 and around 0.01 to 0.015, respectively). The signal strength of the second PPG signal is lower than that of the first PPG signal, even though both PPG signals are measured from a human user. This is because signal strength is affected by factors such as loose wearing of the measurement device on the user's body, body temperature, and skin tone. As shown in the second PPG signal in FIG. 7A, low signal strength can cause noise and distort the pulse shape even if the second PPG signal is measured under low to no motion. The signal strength threshold can be defined based the minimum signal strength which still gives an acceptable pulse shape wherein the peak and valley can still be defined clearly. The signal strength threshold may range from 0.01 to 0.05, for example 0.013 in this sample dataset.

Additionally, in the step 208, the morphology features of the PPG signals are calculated. For example, the morphology features include the rise time and valley-valley jump of each pulse in the red and infrared PPG signals, and the valley-valley jump of each pulse in the red PPG signal.

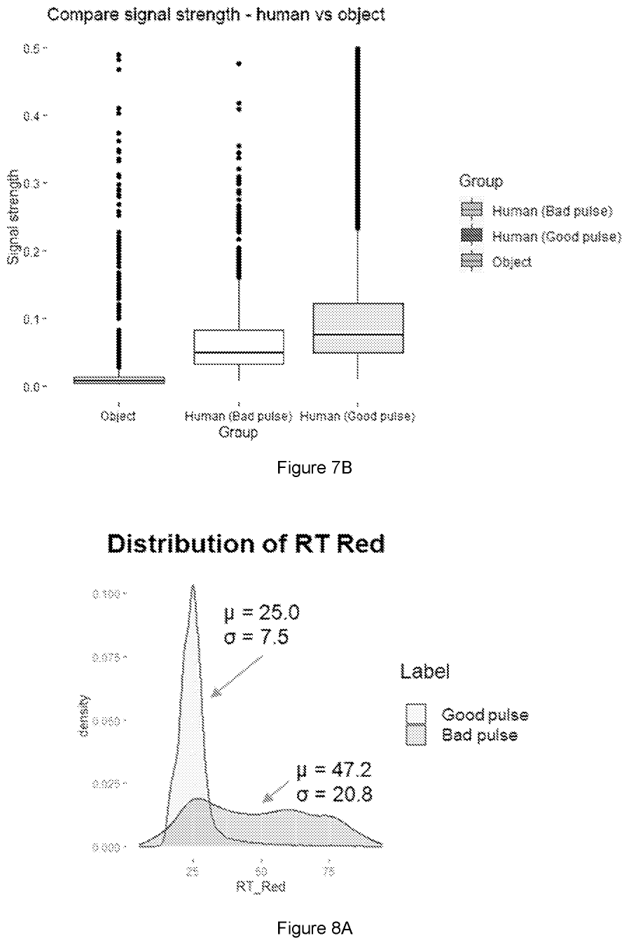

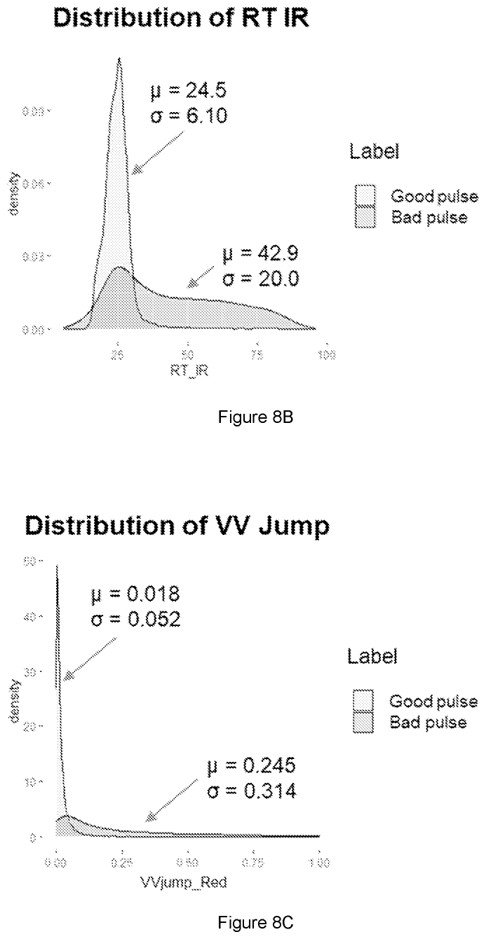

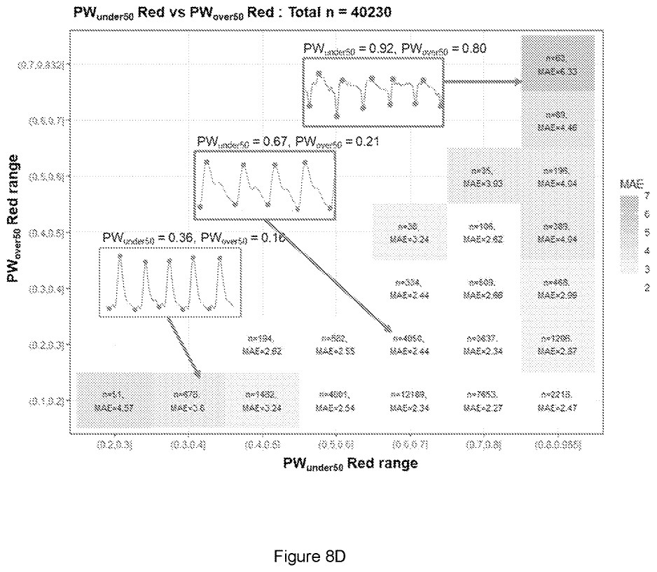

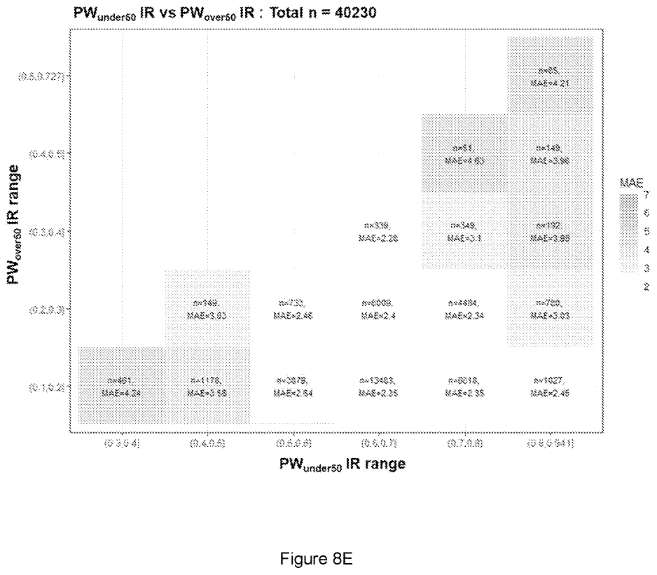

FIGS. 8A to 8C shows the distribution of these morphology features into good and bad pulses. Notably, the standard deviations are wider for the bad pulses, indicating that the bad pulses are more dispersed due to noise randomness. The third data model was trained by logistic regression using a sample dataset of these morphology features. The bad pulse score threshold can be defined by maximizing the F1 score based on similar performance indicators for bad pulse scores. The bad pulse score threshold may range from 0.1 to 0.5, for example 0.14 in this sample dataset.

FIGS. 8D and 8E show the mean absolute error of SpO2 (MAE) at different range of the pulse width features PWₒᵥₑᵣ₅₀ and PW_{under50} derived from red and infrared PPG signals. The MAE is larger in two scenarios s—when PWₒᵥₑᵣ₅₀ is higher than a certain value and when PW_{under50} is lower than a certain value. These trends can be observed in both red and infrared PPG signals and this data is used to train the third data model. Pulses with PWₒᵥₑᵣ₅₀ higher than a threshold or PW_{under50} lower than a threshold are classified as bad pulses and rejected. In this example, thresholds for PWₒᵥₑᵣ₅₀ and PW_{under50} are 0.54 and 0.42, respectively.

In a step 210, the bad pulse score of each pulse in the PPG signals is calculated from the morphology features and third data model. In a step 212, each pulse is looped into a pulse classification process 220 to classify the pulse as good or bad.

For each pulse in the pulse classification process 220, a step 222 compares the motion strength to the motion strength threshold. If the motion strength is below the threshold, the step 222 passes to a step 224. If the motion strength is above the threshold, the step 222 fails and the pulse is classified as a bad pulse in a step 230. The step 224 compares the signal strength to the signal strength threshold. If the signal strength is above the threshold, the step 224 passes to a step 226. If the signal strength is below the threshold, the step 224 fails and the pulse is classified as a bad pulse in the step 230. The step 226 compares the bad pulse score to the bad pulse score threshold. If the bad pulse score is below the score threshold, the step 226 passes to a step 228a. If the bad pulse score is above the score threshold, the step 226 fails and the pulse is classified as a bad pulse in the step 230. The step 228a compares the pulse width over 50% of amplitude (PW_{over50%}) with the threshold. If PW_{over50%} is below the threshold, the step 228a passes to a step 228b. If PW_{over50%} is above the threshold, the step 228a fails and the pulse is classified as a bad pulse in the step 230. The step 228b compares the pulse width under 50% of amplitude (PW_{under50%}) with the threshold. If PW_{under50%} is below the threshold, the step 228b fails and the pulse is classified as a bad pulse in the step 230. If PW_{under50%} is above the threshold, the step 228b passes to a step 232 where the pulse is classified as a good pulse. It will be appreciated that the steps 222, 224, 226, 228a, and 228b may be performed in any sequence.

The pulse verification process 200 thus keeps the good pulses in the PPG signals and the refined PPG signals are used to calculate the modulation ratio and subsequently measure the SpO2 level. In some embodiments, the measured SpO2 level is used to further verify the PPG signals in the pulse verification process 200. More specifically, the measurement device incorporating the method 100 is used to measure the heart rate and SpO2 level from the PPG signals. The measured heart rate and measured SpO2 level are compared to reference heart rate and reference SpO2 level that are measured from a reference device such as a finger pulse oximeter. The measurement device and reference device are communicative with each other to perform this comparison.

The heart rates and SpO2 levels are measured over a set of time periods, such as 4-second periods. For each time period, the heart rate error is calculated by the absolute difference between the measured and reference heart rates, and the SpO2 error is calculated by the absolute difference between the measured and reference SpO2 levels. The pulses of a PPG signal in a time period are classified as good pulses if the SpO2 error is up to 2 standard deviations and the heart rate error is up to 30 bpm. The pulses of a PPG signal in a time period are classified as bad pulses if the SpO2 error is more than 2 standard deviations or the heart rate error is more than 30 bpm.

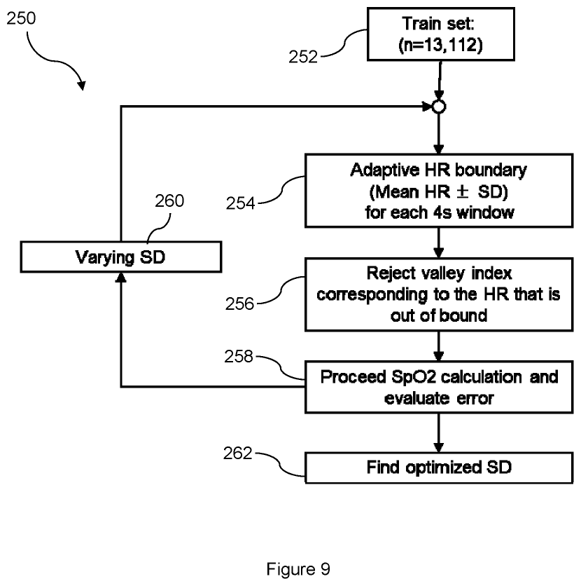

The SpO2 error may be used to optimize the heart rate boundary and make it adaptive to the SpO2 measurements. FIG. 9 illustrates an exemplary iterative process 250 for optimizing the heart rate boundary. In a step 252, a sample dataset is input in the iterative process 250. The sample dataset includes heart rate data from 13,112 time periods of 4 seconds each. In a step 254, for each time period, the heart rate boundary is defined as within a standard deviation from the mean heart rate (such as within 10 to 30 bpm). In a step 256, pulses are rejected if the corresponding heart rates are outside of the heart rate boundary. In a step 258, the resulting PPG signals after rejecting the bad pulses are used to calculate the modulation ratio and measure the SpO2 level. The SpO2 error is also measured in comparison with the reference SpO2 level. In a step 260, the standard deviation is varied and the steps 254, 256, and 258 are repeated iteratively based on the varying standard deviation. The standard deviation is varied until an optimized standard deviation can be found in a step 262. The optimized standard deviation is selected based on the lowest SpO2 error and the lowest number of rejected pulses.

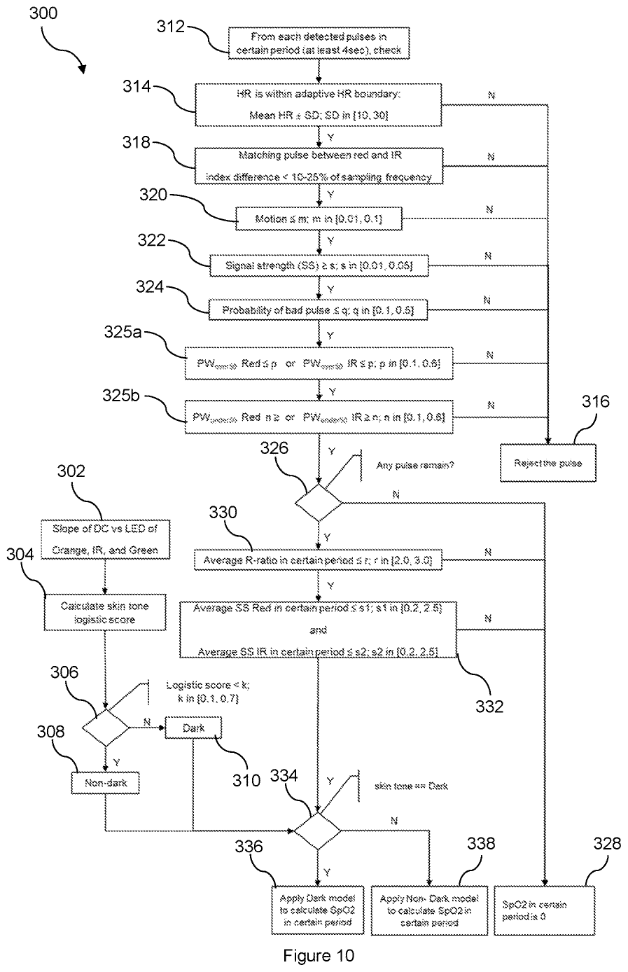

The pulse verification process 200 thus helps to exclude bad or poor quality pulses from the PPG signals, improving the overall quality of the PPG signals that are used to calculate the modulation ratio and subsequently measure or predict the SpO2 level from the selected second data model. An exemplary embodiment of the method 100 including the pulse verification process 200 is shown in FIG. 10 as a method 300 for measuring SpO2 level in a user's blood.

In a step 302, the gradients from green, orange, and infrared PPG signals are calculated. In a step 304, the logistic score is calculated from the gradients and the first data model. In a step 306, the logistic score is compared to the skin tone threshold. If the logistic score is below the skin tone threshold, the user's skin tone is predicted to be non-dark (step 308). If the logistic score is equal to or above the skin tone threshold, the user's skin tone is predicted to be dark (step 310).

In a step 312, pulses in the PPG signals are detected within a time period of at least 4 seconds for verification by the pulse verification process 200. A step 314 checks whether the heart rate is within the heart rate boundary. If the heart rate is outside of the boundary, the step 314 fails and the pulse is rejected in a step 316. If the heart rate is within the boundary, the step 314 passes to a step 318 which checks for matching pulses between the red and infrared PPG signals. If the corresponding valley pairs in the PPG signal do not occur within an allowable time limit, the valley pairs are equal to or above the matching difference threshold and would be considered as unsynchronized, the step 318 fails and the PPG signals in that time period are rejected in the step 316. If the corresponding valley pairs in the PPG signal occur within the allowable time limit, i.e. below the matching difference threshold, the valley pairs would be considered as synchronized, the step 318 passes to a step 320 which compares the motion strength of each pulse to the motion strength threshold. If the motion strength is equal to or above the motion strength threshold, the step 320 fails and the pulse is rejected in the step 316. If the motion strength is below the motion strength threshold, the step 320 passes to a step 322 which compares the signal strength of each pulse to the signal strength threshold. If the signal strength is below or equal to the signal strength threshold, the step 322 fails and the pulse is rejected in the step 316. If the signal strength is above the signal strength threshold, the step 322 passes to a step 324 which compares the bad pulse score of each pulse to the bad pulse score threshold. If the bad pulse score is equal to or above the bad pulse score threshold, the step 324 fails and the pulse is rejected in the step 316. If the bad pulse score is below the probability of bad pulse score threshold, the step 324 passes to a step 325a. If PW_{over50%} is above the threshold, the step 325a fails and the pulse is rejected in the step 316. If PW_{under50%} is below the threshold, the step 325a passes to step 325b. If PW_{under50%} is below the threshold, the step 325b fails, and the pulse is rejected in the step 316. If PW_{under50%} is above the threshold, the pulse is classified as a good pulse and the step 325b passes to a step 326. It will be appreciated that the steps 314, 318, 320, 322, 324, 325a, and 325b may be performed in any sequence.

The step 326 determines whether there are any pulses remaining in the PPG signals after the pulse verification process 200. If there are no pulses remaining, the step 326 proceeds to a step 328 where the SpO2 level for the time period cannot be measured. If there are pulses remaining, these are good quality pulses and the step 326 proceeds to a step 330. The step 330 checks whether the modulation ratio of the PPG signals is equal to or more than the threshold value. If not, the PPG signals are rejected and the SpO2 level cannot be measured (step 328). If yes, the step 330 proceeds to a step 332 which checks whether the signal strengths from the red and infrared PPG signals are equal to or less than the threshold values. If not, the PPG signals are rejected and the SpO2 level cannot be measured (step 328). If yes, the step 332 proceeds to a step 334.

The step 334 selects the second data model based on the user's skin tone determined in the step 306. If the user's skin tone is dark, the second data model for dark skin tone is selected and used to predict the SpO2 level for the time period from the modulation ratio (step 336). If the user's skin tone is non-dark, the second data model for non-dark skin tone is selected and used to predict the SpO2 level for the time period from the modulation ratio (step 338).

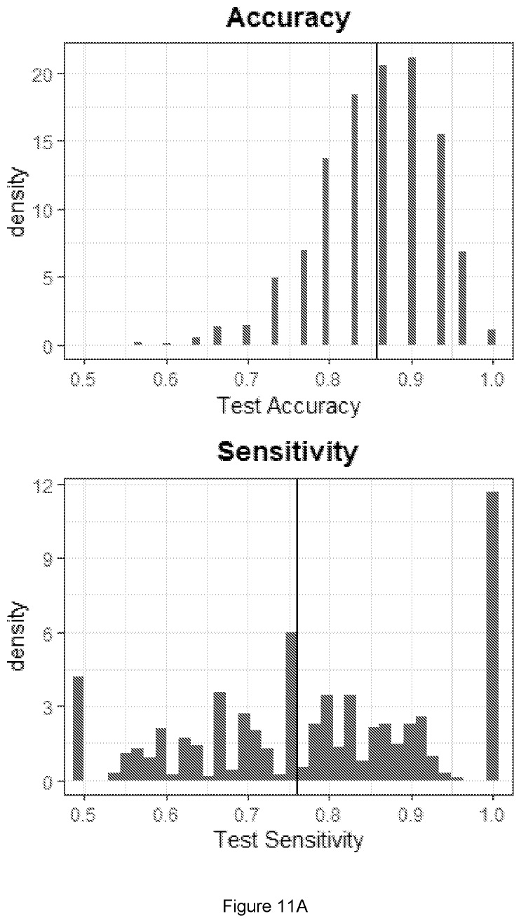

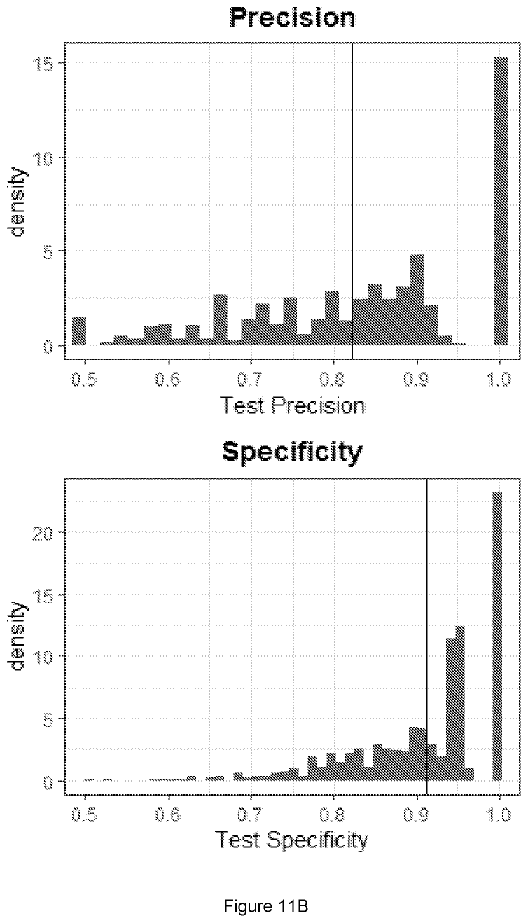

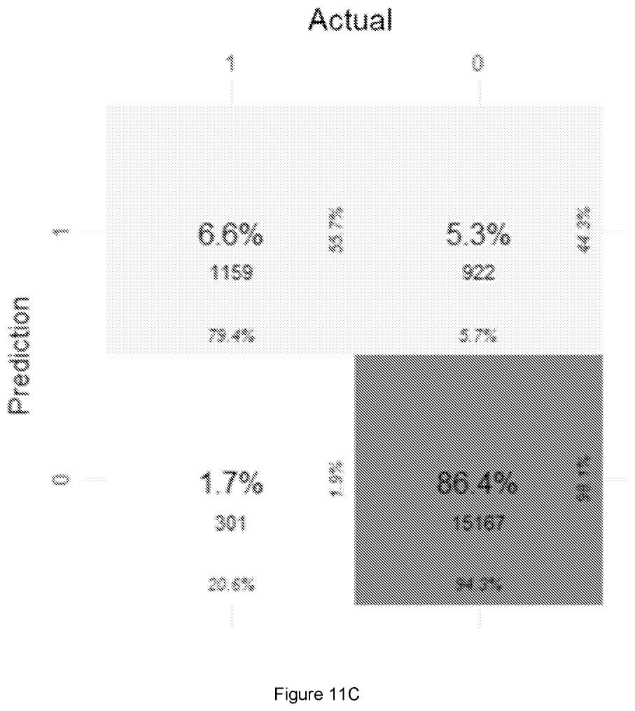

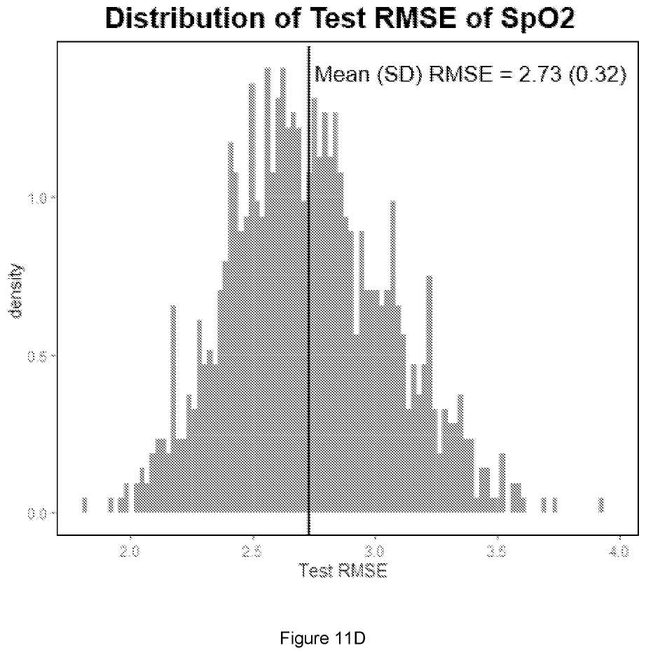

The method 300 was tested using the 98 skin tone datapoints from the 49 subjects described above. The combination of green, orange, and infrared light was tested in 1000 iterations. In each iteration, 15 subjects (i.e. 30 skin tone datapoints) were randomly selected as the test dataset and the other 34 subjects (i.e. 68 skin tone datapoints) form the training dataset. The training dataset was used to train the first data model using logistic regression and the trained first data model was used to evaluate performance on the test dataset in determining skin tones. After completing the 1000 iterations, the average values and standard deviations of the four performance indicators from the test dataset were calculated as follows and their distributions are shown in FIGS. 11A and 11B.
Accuracy: Average 0.86 with standard deviation of 0.07.
Precision: Average 0.82 with standard deviation of 0.16.
Sensitivity: Average 0.76 with standard deviation of 0.17.
Specificity: Average 0.91 with standard deviation of 0.09.

The pulse verification process 200 was able to classify the good and bad pulses in the PPG signals. The performance of the pulse verification process 200 was evaluated on a sample of 17,549 pulses, as shown in the matrix in FIG. 11C. The good pulses are defined as “0” and the bad pulses are defined as “1”. The good and bad pulses predicted by the pulse verification process 200 were compared to target references of good and bad pulses that were classified independently. In one example, the target references of good and bad pulses are obtained by visual observation. A good pulse would have well-defined systolic and/or diastolic peaks regardless of whether the systolic and diastolic waves are clearly defined or not. A bad pulse would have indistinguishable systolic and diastolic waves and/or the pulse baseline is not consistent. In another example, the target references of good and bad pulses are obtained with the help of a finger pulse oximeter. The predicted and reference values (from the finger pulse oximeter) of SpO2 error and HR error are compared, and if the comparison difference is small (e.g. SpO2 error±2SD, HR error±30 bpm), the pulse is defined as good. Around 86.4% and 6.6% of the pulses are correctly predicted as good and bad pulses, respectively. The performance indicators are 93.0% accuracy, 55.7% precision, 79.4% sensitivity, and 94.3% specificity.

The method 300 was used on 33 subjects to predict their SpO2 levels based on their skin tone. The predicted SpO2 levels were compared to reference SpO2 levels measured by a reference device such as a finger pulse oximeter. The performance of SpO2 prediction by the method 300 was tested in 1000 iterations. In each iteration, 10 skin tone datapoints were randomly selected as the test dataset and the other 23 skin tone datapoints form the training dataset for training the second data models using regression analysis. The training dataset was split into 2 subsets by skin tone—one for training the second data model for dark skin tone and the other for training the second data model for non-dark skin tone. The test dataset was similarly split into 2 subsets by skin tone. After completing the 1000 iterations, the average value and standard deviation of the root-mean-square-error (RMSE) between the predicted and reference SpO2 levels from the test dataset was calculated as 2.73 and 0.32, respectively. The distribution of the RMSE is shown in FIG. 11D. The RMSE is within the acceptable limit of 3.5% defined by the United States Food and Drug Administration.

In the foregoing detailed description, embodiments of the present disclosure in relation to a method and device for determining skin tone and measuring oxygen saturation in blood are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. A computerized method for measuring oxygen saturation in a user's blood, the method comprising:
measuring a set of photoplethysmography (PPG) signals from one or more different wavelengths of light, each PPG signal measured from a respective wavelength and comprising pulsatile and non-pulsatile components;
calculating, for each PPG signal, a gradient of the non-pulsatile components of the PPG signal with respect to light intensity of the respective wavelength;
determining the user's skin tone from one or more gradients of the set of PPG signals and a first data model;
calculating a modulation ratio from the pulsatile and non-pulsatile components of a pair of PPG signals measured from a pair of different wavelengths of light;
selecting one from a plurality of second data models based on the user's skin tone; and
determining the oxygen saturation in the user's blood from the modulation ratio and the second data model selected for the user's skin tone.

2. The method according to claim 1, wherein the set of PPG signals comprises a plurality of PPG signals, the method comprising:
measuring the plurality of PPG signals from a plurality of different wavelengths of light; and
determining the user's skin tone from the one or more gradients of the plurality of PPG signals and the first data model.

3. The method according to claim 1, wherein the one or more different wavelengths of light define at least two wavelengths in the range of 495 nm to 1000 nm.

4. The method according to claim 3, wherein the at least two wavelengths at least 50 nm apart from each other.

5. The method according to claim 1, wherein the one or more different wavelengths of light define at least two of green, orange, red, and infrared light.

6. The method according to claim 1, wherein the first and second data models are constructed using one or more machine learning algorithms.

7. (canceled)

8. The method according to claim 1, wherein the second data models comprise a model for dark skin tone and a model for non-dark skin tone.

9. (canceled)

10. (canceled)

11. (canceled)

12. The method according to claim 1, further comprising performing a pulse verification process on the pair of PPG signals for calculating the modulation ratio, the pulse verification process for rejecting pulses in the PPG signals that do not satisfy conditions defined in a third data model.

13. The method according to claim 12, wherein the conditions in the third data model comprise a matching difference threshold between the PPG signals within a time period, such that the PPG signals are rejected if they do not meet the matching difference threshold.

14. The method according to claim 12, wherein the conditions in the third data model comprise a motion strength threshold, such that pulses with motion strength above the motion strength threshold are rejected.

15. (canceled)

16. (canceled)

17. (canceled)

18. The method according to claim 12, wherein the conditions in the third data model comprise a heart rate boundary, such that pulses are rejected if the corresponding heart rates are outside of the heart rate boundary.

19. The method according to claim 18, further comprising optimizing the heart rate boundary in an iterative process.

20. (canceled)

21. (canceled)

22. (canceled)

23. (canceled)

24. A computerized method for determining a user's skin tone, the method comprising:
controlling a lighting element to emit one or more different wavelengths of light by incrementing an electric current supplied to the lighting element;
measuring a set of photoplethysmography (PPG) signals from the one or more different wavelengths of light, each PPG signal measured from a respective wavelength and comprising pulsatile and non-pulsatile components;
calculating, for each PPG signal, a gradient of the non-pulsatile components of the PPG signal with respect to light intensity of the respective wavelength, the light intensity being defined by the electric current supplied to the lighting element; and
determining the user's skin tone from one or more gradients of the set of PPG signals and a first data model.

25. The method according to claim 24, wherein the set of PPG signals comprises a plurality of PPG signals, the method comprising:
measuring the plurality of PPG signals from a plurality of different wavelengths of light; and
determining the user's skin tone from the one or more gradients of the plurality of PPG signals and the first data model.

26. The method according to claim 24, wherein the one or more different wavelengths of light define at least one wavelength in the range of 495 nm to 1000 nm.

27. The method according to claim 26, wherein the one or more different wavelengths of light define at least two wavelengths in the range of 495 nm to 1000 nm.

28. The method according to claim 27, wherein the at least two wavelengths are at least 50 nm apart from each other.

29. The method according to claim 24, wherein the one or more different wavelengths of light define at least one of green, orange, red, and infrared light.

30. A measurement device for measuring oxygen saturation in a user's blood, the measurement device comprising:
one or more lighting elements for emitting one or more different wavelengths of light;
one or more photodetectors for detecting the one or more different wavelengths of light; and
a processor configured for performing the computerized method according to claim 1.

31. A non-transitory computer-readable storage medium storing computer-readable instructions that, when executed, cause a processor to perform the computerized method for measuring oxygen saturation in a user's blood according to claim 1.

32. (canceled)

33. (canceled)
